(19) United States
(12) Patent Application Publication
(10) Pub. No.: US 2022/0296786 A1
(43) Pub. Date: Sep. 22, 2022

(54) FORMULATIONS AND PROCESSES TO GENERATE REPELLENT SURFACES ON MEDICAL DEVICES

(71) Applicant: SpotLESS Materials Inc., State College, PA (US)

(72) Inventors: Nan SUN, State College, PA (US); Birgitt BOSCHITSCH, State College, PA (US); Tak-Sing WONG, State College, PA (US)

(21) Appl. No.: 17/697,248

(22) Filed: Mar. 17, 2022

Related U.S. Application Data
(63) Continuation of application No. PCT/US2022/020481, filed on Mar. 16, 2022.
(60) Provisional application No. 63/162,261, filed on Mar. 17, 2021.

(51) Int. Cl. A61L 28/00; C09D 5/00; C09D 7/63; C09D 183/04

(52) U.S. Cl. CPC A61L 28/0034; A61L 28/0069; C09D 5/00; C09D 7/63; C09D 183/04; A61L 2420/02; A61L 2400/10

(57) ABSTRACT

Formulations for preparing repellent coatings on surfaces of substrates, such as medical devices, can include a non-cyclic volatile siloxane solvent such as a linear or a branched volatile alkyl (e.g., methyl) siloxane solvent and mixtures thereof. Such formulations include (i) one or more reactive components that can form a bonded layer on a surface in which the bonded layer comprises an array of compounds having one end bound to a surface and an opposite end extending away from the surface; (ii) an acid catalyst; and (iii) the non-cyclic volatile siloxane solvent. The formulation can also include (iv) a lubricant.

### CROSS-REFERENCE TO RELATED APPLICATIONS

This application is a continuation of International Application No. PCT/US2022/020481, filed 16 Mar. 2022, which claims the benefit of U.S. Provisional Application No. 63/162,261, filed 17 Mar. 2021, the entire disclosures of each of which are hereby incorporated by reference herein.

### STATEMENT REGARDING FEDERALLY SPONSORED RESEARCH

This invention was made with government support under Contract No. 2026140 awarded by the National Science Foundation. The government has certain rights in the invention.

### TECHNICAL FIELD

The present disclosure relates to formulations with siloxane solvents and use thereof to form repellent coatings on surfaces of substrates.

### BACKGROUND

Repellent coating formulations are known. See for example, Wang, et al., “Covalently Attached Liquids: Instant Omniphobic Surfaces with Unprecedented Repellency”, Angewandte Chemie International Edition 55, 244-248 (2016); WO 2018/094161; WO 2019/222007 and WO 2021/051036.

However, there is a continuing need to develop formulations to form repellent surface coatings that are simple to apply and environmentally acceptable with a sufficient shelf-life.

### SUMMARY OF THE DISCLOSURE

Advantages of the present disclosure include formulations having a non-cyclic, volatile siloxane solvent such as a linear or a branched volatile alkyl siloxane solvent, e.g., a linear or a branched volatile methyl siloxane solvent, and mixtures thereof. Such solvents are considered to have low reactivity to photochemical reactions. The formulations of the present disclosure can be used to prepare repellent coatings for a wide range of solid surfaces including those composed of one or more polymers, ceramics, glasses, glass-ceramics, porcelains, metals, alloys, composites or combinations thereof.

These and other advantages are satisfied, at least in part, by a formulation comprising: (i) one or more reactive components that can form a bonded layer on a surface in which the bonded layer comprises an array of compounds having one end bound to a surface and an opposite end extending away from the surface; (ii) an acid catalyst; (iii) a solvent comprising a non-cyclic volatile siloxane and mixtures thereof and optionally (iv) a lubricant. Useful acid catalysts include sulfuric acid, hydrochloric acid, phosphoric acid, nitric acid, benzoic acid, acetic acid, ascorbic acid, citric acid, formic acid, lactic acid, oxalic acid, or combinations thereof. Useful non-cyclic volatile siloxane solvents include a linear or a branched volatile alkyl siloxane solvent, e.g., a linear or a branched volatile methyl siloxane solvent, and mixtures thereof. Useful optional lubricants include silicone oils or mineral oils or plant oils or any combination thereof.

Advantageously, the formulation of the present disclosure can have a long shelf-life without substantial deactivation of the reactive components when stored around room conditions in closed containers. For example, formulations of the present disclosure can have a stable shelf-life of at least 1 month, such as at least 2, 3, 4, 5, 6, 9, 12 months etc. A stable shelf-life for a storage period can be determined by measuring a sliding angle of a surface of a glass slide having a repellent coating formed from a given formulation at the end of the storage period in which the formulation is stored in a sealed container and the average sliding angle is no more than 35 degrees for a 20 μL water droplet when measured at 20° C. The formulations of the present disclosure can advantageously have a closed cup flash point of more than about 20° C., or more than about 40° C., or 60° C. In addition, formulations of the present application can have a low VOC level, such as a VOC level of less than 6%, e.g., even less than 2%

An additional advantage of the present disclosure includes a process of forming a repellent coating on a surface from the formulations disclosed herein. The process includes drying a formulation disclosed herein on a surface of a substrate to substantially remove the solvent and to form a bonded layer on the surface. Further, if a lubricant is included in the formulation or subsequently applied, the repellent coating further includes a lubricant layer stably adhered to the bonded layer formed from the lubricant. Advantageously, the formed bonded layer comprises an array of compounds each having one end bound to the surface and an opposite end extending away from the surface. The process can also comprise a step of applying the formulation to the substrate surface prior to drying the formulation on the surface.

The repellent coating can be formed on a wide variety of fixtures and devices such as plastic, ceramic, glass, metals and alloys thereof such as in metal plumbing fixtures, surfaces of glass substrates including mirrors, windshields, windows, camera lenses, surfaces of polymers including medical devices such as ostomy appliances, etc. Advantageously, the formulation of the present disclosure can be applied to reusable and disposable consumer containers and packaging such as for cosmetics, foods, hair and skin care products, oral products including toothpaste, etc. In addition, the repellent coating can be formed on devices that are subject to high temperature cycles such as surfaces of induction and radiant cooktops and stoves and other cooking surfaces, ovens as well as tanks, containers, heat exchangers, such as heat exchangers for processing foods and beverages, etc. Such surface can be composed of one or more ceramics, glasses, glass-ceramics, porcelains, metals, alloys, composites or combinations thereof.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### DETAILED DESCRIPTION OF THE DISCLOSURE

The present disclosure relates to formulations employing non-cyclic volatile siloxane solvent such as a linear or a branched volatile alkyl siloxane solvent. Such solvents are considered to have low reactivity to photochemical reactions and thus are specifically exempted from regulatory lists of volatile organic compounds. Volatile organic compounds (VOC) are organic compounds which participate in photochemical reactions to form ozone. Certain volatile solvents are specifically exempted from regulatory lists of VOCs, i.e., VOC exempt solvents, based on low reactivity. However, we found that certain VOC exempt solvents do not result in a stable formulation for the reactive components of the present disclosure.

Formulation of the present disclosure include reactive component(s) to form the bonded layer on a surface of a substrate with an acid catalyst. We found that only certain solvents can be used for forming a stable formulation having a sufficient shelf-life without substantial deactivation of the reactive components. Advantageously, the formulation of the present disclosure can have a long shelf-life without substantial deactivation of the reactive components when stored around room conditions in closed containers. Shelf life is determined by forming a repellant coating from the formulation on a glass slide after the formulation has been stored in a sealed container at the end of the storage period. A formulation having a stable shelf life for the storage period is one in which an average sliding angle of the surface having the repellent coating formed from the formulation is no more than 35 degrees for a 20 μL water droplet when measured at 20° C. at the end of the period. Such an average sliding angle can be determined by three independent measurements. It may be helpful to also determine any change in the stability of the formulation, which can be carried out by determining the sliding angle when the formulation is initially prepared and again after a certain storage period.

Repellent coatings on surfaces of substrates as disclosed herein can be thermally stable such that the repellent coating on the surface of the substrate can be maintained at a temperature of above 100° C., e.g., above 100° C. to about 300° C., for at least 10 minutes, such as at least 20 minutes, 30 minutes, etc. For example, the surface having the repellent coating can have an average sliding angle for a 20 μL water droplet of no more than about 35°, such as no more than about 30°, 25°, 20°, etc. and even less than about 10° when measured at 20° C., after repeated high temperature cycling.

Repellent coatings on surfaces of substrates as disclosed herein can be formed from a formulation that includes: (i) reactive component(s) to form the bonded layer on a surface of a substrate; (ii) acid catalyst(s); (iii) solvent(s); and optionally (iv) lubricant(s). The reactive component(s) of the formulation are used to form the bonded layer onto the surface of a substrate by allowing them to react with the surface to form an array of compounds on the surface in which each compound has one end covalently bound to the surface and an opposite end extending away from the surface. As such, the bonded layer resembles a brush with linear chains bound to the surface. The acid catalyst facilitates and accelerate formation of the bonding layer at a reduced time and temperature and the solvent can also facilitate formation of the bonding layer. An optional lubricant layer can be stably adhered to the bonded layer primarily through van der Waals interactions to enhance the repellent coating. The lubricant used to form the lubricant layer can be included in the initial formulation applied to the substrate surface or the lubricant can be applied after formation of the bonded layer on the substrate. When included in the formulation or applied to the bonded layer, the lubricant preferably forms a lubricant layer that is stably adhered to the bonded layer. In an aspect of the present disclosure, the formulation includes the optional lubricant. Such a formulation can form a repellent coating comprising a bonded layer with a lubricant layer stably adhered to the bonded layer as an all-in-one formulation.

The bonded layer can be formed directly or indirectly on a surface of a substrate by reacting the reactive components of the formulation directly with functional groups, e.g. hydroxyl groups, acid groups, ester groups, etc., which are on the surface of the substrate. Such functional groups can be naturally present or induced on the substrate such as by treating the surface with oxygen or air plasma or corona discharge or by heating under the presence of air or oxygen, etc.

Useful reactive components for formulations of the present disclosure include, for example, reactive components that have one end that bonds to the substrate surface, e.g., covalently bonds to one or more reactive groups on the surface, to form an assembly of compounds. Such reactive components preferably have a chain length of at least 3 carbons. Other useful reactive components include polymerizable monomers that can react to form an array of linear polymers having ends anchored to the surface and opposite ends extending away from the surface. To increase the speed of forming a coating, the reactive components of the formulation are selected to undergo a condensation reaction with loss of a small molecule such as water, an alcohol, etc. which can be readily removed to drive the reaction to more or less completion under ambient temperatures and pressures. Preferably the linear polymers, with one end attached to the surface and the other extending away from the surface, do not form covalent bonds with the adjacent linear polymers or crosslink, such as crosslinks with the adjacent linear polymers (e.g., the linear polymers form a brush-like structure). A lack of crosslinking allows the chains and ends extending away from the surface higher mobility to further enhance the repellency of the repellent coating.

Useful reactive components for formulations of the present disclosure include, for example, low molecular weight silanes or siloxanes that have one or more hydrolysable groups. Such silanes or siloxanes have a molecular weight of less than about 1,500 g/mol such as less than about 1,000 g/mol and include a monoalkyl or mono-fluoroalkyl phosphonic acid such as 1H,1H,2H,2H-perfluorooctane phosphonic acid, an alkoxysilane such as a mono-alkoxy silane, e.g., an alkyl, fluoroalkyl and perfluoroalkyl mono-alkoxy silane, trimethylmethoxysilane; a di-alkoxy silane, e.g., a dialkyl di-alkoxy silane, such as a C₁-8 dialkyldialkoxy silane e.g., dimethyldimethoxysilane, dimethoxy(methyl)octylsilane, a di-alkoxy, diphenyl silane, diethyldiethoxysilane, diisopropyldimethoxysilane, di-n-butyldimethoxysilane, diisobutyldimethoxysilane, diisobutyldiethoxysilane, isobutylisopropyldimethoxysilane, dicyclopentyldimethoxysilane, a di-alkoxy, fluoroalkyl silane or perfluoroalkyl silane, dimethoxy-methyl(3,3,3-trifluoropropyl)silane, (3,3,3-trifluoropropyl)methyl dimethoxysilane, a alkyltrimethoxysilane, a tri-alkoxy silane, e.g., a perfluoroalkyl-tri-alkoxy silane, trimethoxy(3,3,3-trifluoropropyl)silane, trimethoxymethylsilane, 1H, 1H,2H,2H-perfluorodecyltrimethoxysilane, 1H, 1H,2H,2H-perfluorodecyltriethoxysilane, nonafluorohexyltrimethoxysilane, nonafluorohexyltriethoxysilane, (tridecafluoro-1,1,2,2-tetrahydrooctyl)trimethoxysilane, tridecafluoro-1,1,2,2-tetrahydrooctyl)triethoxysilane, heptadecafluoro-1,1,2,2-tetrahydrodecyl)trimethoxysilane, (heptadecafluoro-1,1,2,2-tetrahydrodecyl)triethoxysilane, a chlorosilane, e.g., octyldimethylchlorosilane, a dichlorosilane, e.g., di ethyl dichlorosilane, di-n-butyl dichlorosilane, diisopropyldichlorosilane, dicyclopentyldichlorosilane, di-n-hexyldichlorosilane, dicyclohexyldichlorosilane, di-n-octyldichlorosilane, 3,3,3-trifluoropropyl)methyl dichlorosilane, nonafluorohexylmethyldichlorosilane, (tridecafluoro-1,1,2,2-tetrahydrooctyl)methyldichlorosilane, (heptadecafluoro-1,1,2,2-tetrahydrodecyl)methldichlorosilane, (3,3,3-trifluoropropyl)dimethylchlorosilane, nonafluorohexyldimethylchlorosilane, tridecafluoro-1,1,2,2-tetrahydrooctyl)dimethylchlorosilane, (heptadecafluoro-1,1,2,2-tetrahydrodecyl)dimethylchlorosilane, a trichlorosilane, e.g., (tridecafluoro-1,1,2,2-tetrahydrooctyl)trichlorosilane, (3,3,3-trifluoropropyl)trichlorosilane, nonafluorohexyltrichlorosilane, (heptadecafluoro-1,1,2,2-tetrahydrodecyl)trichlorosilane, an amino silane, e.g., nonafluorohexyltris(dimethyamino)silane, etc.

The alkoxy groups of such reactive components can be C₁-4 alkoxy groups such as methoxy (—OCH₃), ethoxy (—OCH₂CH₃) groups and the alkyl groups of such reactive components can have various chain lengths, e.g., of C₁₋₃₀, such as C₃₋₃₀. The alkyl groups of such reactive components that form linear polymers generally have a lower alkyl group, e.g., C₁₋₁₆, such as C₁₋₈. The alkyl groups in each case can be substituted with one or more fluoro groups forming fluoroalkyl and perfluoroalkyl groups of C₁₋₃₀, C₃₋₃₀, C₁₋₁₆, C₁₋₈, etc. chains such as a fluoroalkyl or perfluoroalkyl alkoxysilane, a difluoroalkyl or diperfluoroalkyl di-alkoxy silane, a fluoralkyl or perfluoralkyl tri-alkoxy silane having such chain lengths.

The bonded layer can be formed from the formulation by reacting the reactive components of the formulations directly with exposed hydroxyl groups or other reactive groups on the surface of a substrate to form an array of linear compounds having one end covalently bound directly to the surface through the hydroxyl groups or other reactive groups on the surface of a substrate. Alternatively, the bonded layer can be formed by polymerizing one or more of a silane monomer directly from exposed hydroxyl groups or other reactive groups on the surface of a substrate to form an array of linear polysilanes or polysiloxanes or a combination thereof covalently bound directly to the surface through the hydroxyl groups or other reactive groups on the surface of a substrate. Preferably the linear polymers, with one end attached to the surface and the other extending away from the surface, do not form covalent bonds or crosslink with the neighboring linear polymers (e.g., forms brush-like structures).

The bonded layer can have a thickness of less than about 1000 nm. In some cases, the thickness of the bonded layer can be less than about 500 nm, less than about 100 nm or even less than about 10 nm, e.g. from about 1 or 5 nm to about 500 nm.

One or more catalysts can be included in the formulations of the present disclosure. As used herein a catalyst refers to one or more catalysts. A catalyst can facilitate and accelerate formation of the bonding layer. Useful catalysts that can be included in the formulation include acid catalysts such as sulfuric acid, hydrochloric acid, phosphoric acid, nitric acid, benzoic acid, acetic acid, ascorbic acid, citric acid, formic acid, lactic acid, oxalic acid, or combinations thereof. In some embodiments, the catalyst does not include a catalyst containing a transition metal such as platinum since such catalysts tend to increase costs and remain in a formed coating including such catalysts.

The formulation of the present disclosure also includes a solvent, carrier or medium which can be a single solvent or multiple solvents such as a solvent system, collectively referred to herein as a solvent. Solvents of the present disclosure facilitate formation of the bonding layer and, when the lubricant is present in the formulation, the infusion of the lubricant within the bonding layer during formation of the repellent coating on the surface. Preferably, the solvent should have a relatively low boiling point and relatively high vapor pressure for ease of evaporating the solvent from the formulation when forming the repellent coating therefrom. Preferably, the solvent is exempt from VOC restrictions and does not contribute to ozone depletion. In an embodiment, the solvent of formulations of the present disclosure can have a boiling point at atmospheric pressure of no more than about 235° C., or no more than 200° C., or no more than 155° C., such as no more than about 100° C. and no more than about 82.5° C. and even no more than about 60° C. In other embodiments, the solvent of formulations of the present disclosure can have a vapor pressure of between about 30 kPa at 25° C. and about 0.05 kPa at 25° C. For example, hexamethyldisiloxane has a vapor pressure of about 4.6 kPa at 25° C., decamethyltetrasiloxane and dodecamethylpentasiloxane have a vapor pressure of around 0.13 kPa at 25° C. Solvents with higher boiling points and lower vapor pressure can be used but tend to inhibit the rate of drying and/or may need to be removed by application of a reduced atmospheric pressure or higher temperature to remove the solvent.

Preferably, the solvent should have a relatively high closed-cup flash point to reduce the flammability of the overall formulation. Specifically, flammable liquids are classified by the National Fire Protection Association (NFPA) as Class I with flash points below 100° F. (37.8° C.); whereas combustible liquids are classified as Class II and Class III with flash points in between 100° F. (37.8° C.) and 200° F. (93° C.). In an aspect, the formulations of the present disclosure can have a closed-cup flash point of more than about 20° C., or more than about 40° C., or 60° C., such as more than about 80° C. and even a closed-cup flash point of more than about 100° C. In an embodiment, formulations of the present disclosure can have a closed cup flash point of from about 35° C. to about 130° C., e.g., from about 40° C. to about 125° C. For example, dodecamethylpentasiloxane has a closed-cup flash point of about 75° C. and low viscosity polydimethylsiloxane (3 cSt) has a closed-cup flash point of about 100° C. Flash points of solvents and formulations of the present disclosure can be measured by ASTM D93 Closed Cup Flash Point protocol or an equivalent protocol.

Useful solvents that can be included in the formulation of the present disclosure can include one or more of a non-cyclic volatile siloxane solvent such as a linear or a branched volatile alkyl siloxane solvent and mixtures thereof. Such solvents can have the following formula (R₂ₓ₊₂SiₓOₓ₋₁), in which x is 2 to about 10, e.g., x is 2 to about 8, R represents a radical bound to Si and can be the same or different and can be a H and/or a linear or branched C₁₋₈ alkyl such as a linear or branched C₁₋₄ alkyl, e.g., methyl or ethyl. Since many linear or a branched volatile alkyl siloxane solvents tend to be mixtures, the value of x in the formula of (R₂ₓ₊₂SiₓOₓ₋₁) is an average between 2 to about 10. Further, it is preferable that linear or a branched volatile alkyl siloxane solvents have a viscosity of no more than about 4 cSt (as measured at 25° C.). Such non-cyclic volatile siloxane solvents include, for example, linear volatile methyl siloxanes such as dimethyl silicones and siloxanes, e.g., hexamethyldisiloxane, octamethyltrisiloxane, decamethyltetrasiloxane, dodecamethylpentasiloxane, low viscosity polydimethylsiloxane (3 cSt, at 25° C.), etc. and branched volatile methyl siloxane solvents such as 1,1,1,3,5,5,5-Heptamethyl-3-[(trimethylsilyl)oxyl]-trisiloxane, 1,1,1,5,5,5-Hexamethyl-3,3,-bis [(trimethylsilyl)oxy]-trisiloxane, and mixtures thereof.

Further, in certain aspects, the solvent in the formulation of the present disclosure comprises one or more non-cyclic volatile siloxane as at least 50 wt %, e.g., in at least 80 wt % of the total amount of the solvent. In certain embodiments, the solvent comprises as at least 90 wt %, 95 wt %, 97 wt %, 99 wt % and up to 100% of one or more non-cyclic volatile siloxanes with only trace amounts, if any, of other solvents. Cyclic siloxanes do not appear to form stable formulations with the reactive components of the present disclosure. High amounts of lower ketones; e.g., a C₃₋₈ ketones; lower, primary alcohols, e.g., primary C₁₋₈ alcohols; and lower carbonates (C₃-C₁₂) also do not appear to form stable formulations with the reactive components of the present disclosure when such solvents are combined with non-cyclic volatile siloxane solvents. Hence, such cyclic siloxanes, primary alcohols, ketones and carbonates are preferably not included in the formulation in an amount of more than 20 wt %, and preferably less than 10 wt %, 5 wt %, 3 wt %, and less than 1 wt % or at a level of an impurity if at all.

Further, the formulations of the present disclosure preferably do not include a substantial amount volatile organic compounds as solvents. VOCs, as defined by the U.S. Environmental Protection Agency and adopted herein, include any compound of carbon, excluding carbon monoxide, carbon dioxide, carbonic acid, metallic carbides or carbonates, and ammonium carbonate, which participates in atmospheric photochemical reactions. Such VOCs include, for example, ethanol, isopropanol, hexane, benzene, toluene, xylene, chloroform, formaldehyde. Such VOCs are preferably not included in the formulation in an amount of more than about 20 wt %, and preferably less than about 10 wt %, 5 wt %, 3 wt %, and less than about 1 wt % or at a level of an impurity, if at all.

The formulation of the present disclosure can also include a lubricant or combination of lubricants, collectively referred to herein as a lubricant. In addition or in the alternative, a lubricant can be applied to a bonded layer after forming the bonded layer. In either case, when part of the initially applied formulation or applied subsequently, the lubricant preferably forms a lubricant layer stably adhered to the bonded layer. To form a stably adhered lubricant layer to a bonded layer which in turn is formed from the reactive components of the formulation, a lubricant should have strong affinity to the bonded layer and/or the substrate so that the lubricant can fully wet the surface (e.g., result in an equilibrium contact angle of less than about 5°, such as less than about 3°, about 2°, or less than about 1°, or about 0°) and stably adhere on the surface. Further, since certain surfaces of substrates and repellent coating thereon can be subjected to temperatures above 100° C., the lubricant preferably has a low vapor pressure under atmospheric pressure. In addition, the lubricant should be mobile in the formed repellent coating and thus it is preferable that the lubricant not substantially react, if at all, with the reactive components in the formulation. A stably adhered lubricant to the bonded layer is believed due primarily to van der Waals forces, not through covalent bonding to the bonding layer. In certain embodiments, lubricants for the present disclosure do not have groups that would react with the reactive components of the formulation.

Further, a stably adherent lubricant is distinct from a lubricant placed on a surface, or modified surface, that does not wet the surface (e.g. forms an equilibrium contact angle of greater than 10°) and/or simply slides off the surface within minutes or shorter periods when the surface is raised to a sliding angle of up to 90°. A lubricant layer stably adhered to a bonded layer is one that substantially remains (greater than about 80%) and covering the bonded layer for at least one hour (or longer periods such as several hours and days and months) even when the surface substrate is at a 90° from horizontal and at a temperature of 25° C. In certain aspects, a stable lubricant layer is one that will not be displaced by a lubricant-immiscible fluid placed on the repellent coating having a lubricant layer.

A lubricant useful for formulations and repellent coatings of the present disclosure should have a sufficient viscosity yet be relatively mobile to facilitate repellence of the coating system at temperatures intended for use with the substrate having the repellent coating. Such temperatures can range from about −50° C. to about 300° C. In addition, the surface of the substrate and repellent coating thereon can be subjected to high temperature cycling of above and below 100° C. and the cycle repeated multiple times. As such, a lubricant should preferably have a viscosity of at least about 5 cSt (as measured at 25° C.) such as at least about 6 cSt, 7 cSt, 8 cSt, 9 cSt, 10 cSt, 15 cSt, 20 cSt, 30 cSt, 40 cSt, 50 cSt, etc. and any value therebetween. Further, so that the lubricant can be mobile at certain temperatures in which the repellent coating can be used, a lubricant should preferably have a viscosity of no more than about 1,500 cSt as measured at 25° C., such as no more than about 1,200 cSt, 1,100 cSt, 1,000 cSt, 900 cSt, 850 cSt, etc., as measured at 25° C., and any value therebetween. In an embodiment, a lubricant for a formulation of the present disclosure can have viscosity ranging from about 5 cSt to about 1500 cSt, such as from about 5 cSt, 6 cSt, 7 cSt, 8 cSt, 9 cSt, 10 cSt, 15 cSt, 20 cSt, 30 cSt, 50 cSt, 100 cSt, etc. to about 1500 cSt, 1200 cSt, 1000 cSt, 800 cSt, 350 cSt, 200 cSt, 150 cSt, etc., as measured at 25° C., and any value therebetween. For high temperature uses, the repellent coating can have a lubricant with an even higher viscosity at 25° C. since the viscosity of such a lubricant would be less at the higher use temperature. Further, lubricant densities of less than about 2 g/cm³ would be preferable at temperature range from 15° C. to 25° C.

A lubricant included in the formulation of the present disclosure can be one or more of an omniphobic lubricant, a hydrophobic lubricant and/or a hydrophilic lubricant. The lubricant can include a fluorinated oil or a silicone oil (such as food grade silicone oil) or a mineral oil or a plant oil. Other lubricants that can be used include fluorinated or perfluoropolyether, perfluoroalkylamine, perfluoroalkylsulfide, perfluoroalkylsulfoxide, perfluoroalkylether, perfluorocycloether oils and perfluoroalkylphosphine and perfluoroalkylphosphineoxide oils as well as mixtures thereof. Preferable, the lubricant is chosen to have a strong chemical affinity to the particular bonding layer and/or substrate so that the lubricant can fully wet and stably adhere to the surface via the bonding layer. For example, perfluorinated oils such as a perfluoropolyether (e.g., Krytox oil) can fully wet and stably adhere to a polymeric siloxane and/or silane bonding layer including fluorinated alkyl silanes such as perfluorinated alkyl silanes. Such a bonding layer can be formed from reactive fluoroalkyl silanes in a formulation that reacts with functional groups on a surface of a substrate. Silicone oil or plant oil can fully wet and stably adhere to a bonded layer comprised of an array of linear polydimethylsiloxane (PDMS), for example. Hydroxy polydimethylsiloxane can also fully wet and stably adhere to a bonded layer comprised of an array of linear polydimethylsiloxane (PDMS), for example, but a hydroxy polydimethylsiloxane lubricant would preferably be applied separately from the formulation since it can react with the reactive components of the formulation. A linear polydimethylsiloxane bonding layer can be formed from polymerizing dimethyldimethoxysilane from a surface of a substrate. Mineral oils or plant oils can fully wet and stably adhere to a bonding layer including an array of alkyl silanes which can be formed from alkyltrichlorosilanes or alkyltrimethoxysilanes. The alkyl groups on such alkylsilanes can have various chain lengths, e.g., alkyl chains of C₁₋₃₀.

Other lubricants that will be compatible with bonding layers composed of alkylsilanes with various chain lengths and polysiloxanes polymerized from one or more dialkyldialkoxysilanes such as dimethyldimethoxysilane include, for example, alkane oils, and plant oils such as a vegetable oil, avocado oil, algae extract oil, olive oil, palm oil, soybean oil, canola oil, castor oil, rapeseed oil, corn oil, peanut oil, coconut oil, cottonseed oil, palm oil, safflower oil, sesame oil, sunflower seed oil, almond oil, cashew oil, hazelnut oil, macadamia oil, Mongongo nut oil, pecan oil, pine nut oil, peanut oil, walnut oil, grapefruit seed oil, lemon oil, orange oil, amaranth oil, apple seed oil, argan oil, avocado oil, babassu oil, ben oil, borneo tallow nut oil, cape chestnut oil, carob pod oil, camellia seed oil, cocoa butter, cocklebur oil, cohune oil, grape seed oil, Kapok seed oil, Kenaf seed oil, Lallemantia oil, Manila oil, Meadowfoam seed oil, macadamia nut oil, mustard oil, Okra seed oil, papaya seed oil, Pequi oil, poppyseed oil, pracaxi oil, prune kernel oil, quinoa oil, ramtil oil, rice bran oil, rapeseed oil, sesame oil, safflower oil, Sapote oil, Shea butter, squalene, soybean oil, tea seed oil, tigernut oil, tomato seed oil, liquid terpenes, and other similar bio-based oils or synthetic oil, e.g., polycitronellol acetate etc. The plant-based oils can be used alone or with other lubricants or as a mixture of plant-based oils alone or with other lubricants.

Other components can be included in the formulations of the present disclosure such as a fragrance, i.e., a substance that emits a pleasant odor, and/or a masking compound, i.e., a substance that masks the odors of other ingredients. A fragrance includes, for example, a natural or synthetic aroma compound or an essential oil such as a lemon oil, bergamot oil, lemongrass oil, orange oil, coconut oil, peppermint, oil, pine oil, rose oil, lavender oil or any combination of the foregoing. As an example, the fragrance added to the formulation of the present disclosure can have a smell of lemon, or rose, or lavender, or coconut, or orange, or apple, or wood, or peppermint, etc. One or more fragrance or masking compound can be added to a formulation of the present disclosure as is, e.g., without dilution, and can be added in a range of about 0.0005 parts to about 10 parts, e.g. from about 0.01 to about 5 parts, by weight in place of the solvent. In certain aspects, the fragrance and/or masking compound is soluble in alcohols and siloxanes.

In certain embodiments, the concentrations of various components on a weight bases in formulations of the present disclosure can include the ranges provided in the tables below:

TABLE A1
Formulations without lubricant

| Component | Approximate Concentration Range | |
|---|---|---|
| Reactive component | 1-20 | wt % |
| Solvent | 78-99 | wt % |
| Acid Catalyst | 0.01-2 | wt % |

TABLE A2
Formulations without lubricant

| Component | Approximate Concentration Range | |
|---|---|---|
| Reactive component | 1-20 | wt % |
| Solvent | 65-97 | wt % |
| Acid Catalyst | 2-15 | wt % |

TABLE B1
Formulations with lubricant

| Component | Approximate Concentration Range | |
|---|---|---|
| Reactive component | 1-20 | wt %, |
| Solvent | 28-99 | wt %, |
| Acid Catalyst | 0.01-2 | wt % |
| Lubricant | 0.05-50 | wt % |

TABLE B2
Formulations with lubricant

| Component | Approximate Concentration Range | |
|---|---|---|
| Reactive component | 1-20 | wt %, |
| Solvent | 15-97 | wt %, |
| Acid Catalyst | 2-15 | wt % |
| Lubricant | 0.05-50 | wt % |

In an aspect of the present disclosure, a repellent coating can be formed from a fluorinated alkyl silane and/or a fluorinated lubricant onto a substrate, such as one or more perfluoroalkyl silanes, and one or more perfluorinated oils. For example, one or more C₂-C₁₇ fluorinated or perfluorinated alkyl silane reactive components (e.g., about 1 wt % to about 10 wt %) can be combined in a formulation with an acid catalyst and solvent and the formulation applied and dried on a substrate surface to form a bonded layer of the fluorinated or perfluorinated alkyl silane. One or more fluorinated or perfluoropolyether lubricants can then be applied to the bonded layer to form a lubricant layer stably adhered to the bonded layer. Such a coating can be formed on a variety of substrate surfaces such as those composed of one or more polymers, ceramics, glasses, glass-ceramics, porcelains, metals, alloys, composites or combinations thereof and for a variety of devices including windows, camera lenses, medical devices, heat exchanger surfaces, reusable and disposable consumer containers and packaging such as for cosmetics, foods, hair and skin care products, oral products including toothpaste, for example.

Repellent coatings prepared from formulations of the present disclosure can repel and resist adherence of broad range of liquids and solids including but not limited to water, ice, soapy water, hard water, minerals, plastics, debris, bacteria, residues, such as residue from food stuffs, dairy products, proteins, fats, yeast, biological fluids, urine, feces, blood, etc.

In practicing certain aspects of the present disclosure, it is preferable to form a repellent coating on a substrate with a relatively smooth surface. In some embodiments, the substrate surface has an average roughness (Ra) at a microscale level, e.g., Ra of less than a few microns, and preferably less than a few hundred nanometers, or even less than a few nanometers. Advantageously, the surface of a substrate to which a repellent coating is to be formed thereon is relatively smooth, e.g., the surface has an average roughness Ra of less than about 4 μm, e.g., less than about 2 μm and less than about 1 μm average surface roughness and even less than about 500 nm, e.g., less than about 100 nm, 80 nm, 60 nm, 40 nm 20 nm, 10 nm, etc. average surface roughness.

Average surface roughness can be measured by atomic force microscope (AFM) using tapping mode with a scanning area of 2×2 μm² for measuring average surface roughness in a 0.1-nanometer scale or equivalent technique. Average surface roughness can be measured by Zygo optical profilometer with an area of 100×100 μm² to 500×500 μm² for measuring average surface roughness in a 1-nanometer scale or equivalent technique.

In practicing certain aspects of the present disclosure, the surface of the substrate can be treated to form reactive groups thereon such as hydroxyl groups, such as by applying and removing an alcohol, by oxygen plasma treatment, or by heating under the presence of air or oxygen (for the case of metals). The substrate can include a reactive coupling layer and the repellent coating formed on the surface of the coupling layer.

The substrate surface can be cleaned and dried before applying a formulation of the present disclosure. One example for the cleaning a substrate surface involves the use of a lower alcohol, e.g., ethanol or isopropanol, to rinse the surface. Then the surface can be dried and the formulation applied.

Processes for preparing a repellent coating on a surface of a substrate includes drying a formulation of the present disclosure on a surface of a substrate to substantially remove the solvent, e.g., greater than about 60%, 65%, 70%, 80%, 85%, 90%, 95%, 99% by weight and higher of the solvent can be removed in the drying step. Drying the formulation concentrates the reactive components and causes them to react to form a bonded layer on the surface of the substrate. The reactive components are chosen such that they react with the surface to form an array of compounds each having one end bound to the surface and an opposite end extending away from the surface. Drying the formulation also causes the lubricant to be concentrated and retained within the bonded layer, when present in the initial formulation. The lubricant is thus chosen to have an affinity for the bonded layer and/or surface so that it can form a lubricant layer stably adhered to the surface via the bonded layer.

Repellent coatings on a surface of a substrate can advantageously be formed by drying under relatively low temperatures, e.g., temperatures ranging from about 0° C. to about 80° C. Hence, forming the repellent coating from formulations of the present disclosure can be carried out at from about 5° C. to about room temperature, e.g., 20° C., and at an elevated temperature, e.g., greater than about 25° C., 30° C., 40° C., 50° C., 55° C., 60° C., 70° C., 80° C., etc. Forming the repellent coating can also be advantageously carried out in a relatively short period of time such as in a period of no more than about 120 minutes such as 60 minutes, e.g., no more than about 30 minutes, and no more than 20 minutes, and no more than 10 minutes, and even as short a period of no more than about 5 minutes and no more than about 3 minutes and even no more than 1 minute. Although a vacuum could accelerate drying of the formulation, it is not necessary for the process and drying of formulations of the present disclosure can be carried out at atmospheric pressure, e.g., at about 1 atm. Further, drying and/or applying the formulation of the present disclosure can be carried out in air with relative humidity between 10% to 80% at temperatures from about 5° C. to about 75° C.

Applying formulations of the present disclosure on to a surface of a substrate can be carried-out with liquid-phase processing thereby avoiding complex equipment and processing conditions. Such liquid-phase processing includes, for example, simply submerging the substrate (dip-coating) or applying the formulation on to the substrate surface by wiping, spraying (including aerosol spray), curtain coating and/or spin coating the formulation on to the surface. Other methods of applying formulations of the present disclosure on to a surface of a substrate can be carried out by wiping a towel made of a fabric, paper or similar material, or a sponge or squeegee, infused with the formulation, on the surface to transfer the formulation from the towel, sponge, squeegee to the surface of the substrate. Advantageously, the formulation can be applied to the substrate surface under ambient temperatures and/or atmospheric pressures and in air, e.g., formulations of the present disclosure can be applied on surfaces of substrates in air and at atmospheric pressure. In certain embodiments, the formation of the bonded layer is accelerated in the presence of a catalyst, e.g., an acid catalyst, and water. The water can be either available from the solvent or from the atmosphere or both. Drying the formulation in an atmosphere having some moisture, e.g., an ambient humidity of at least about 10% at 20° C. and atmospheric pressure is preferable from certain of the reactive components. Hence in some embodiments, the formulation of the present disclosure is dried at an ambient humidity of from about 10% to no more than about 80%.

In some instances and under certain conditions, the lubricant layer of a repellent coating can be depleted over time. Advantageously, the lubricant layer can be replenished by applying lubricant, either the same or a different lubricant than used to prepare the repellent coating, to the bonded layer to renew the repellent coating system on the surface of the substrate. The applied lubricant can be in undiluted form when applied to the bonded layer or diluted with medium when applied to the bonded layer. The medium can include water, one or more of a lower ketone, e.g., a C₁₋₈ ketone such as acetone, methyl ethyl ketone, cyclohexanone, a lower alcohol, e.g., a C₁₋₈ alcohol such as methanol, ethanol, isopropanol, a butanol, a lower ether, e.g., a C₁₋₈ ether such as dimethyl ether, diethyl ether, tetrahydrofuran, a lower ester, e.g., a C₁₋₈ ester such as ethyl acetate, butyl acetate, glycol ether esters, a lower halogenated solvent, e.g., a chlorinated C₁₋₈ such as methylene chloride, chloroform, an aliphatic or aromatic hydrocarbon solvent such as hexane, cyclohexane, toluene, xylene, dimethylformamide, dimethyl sulfoxide and any combination thereof. The medium can also include or consist of a volatile organic compound exempt solvent. Such a medium can include, for example, a linear or a branched volatile methyl siloxane solvent. Such solvents include, for example, linear volatile methyl siloxanes such as dimethyl silicones and siloxanes, e.g., hexamethyldisiloxane, octamethyltrisiloxane, decamethyltetrasiloxane, dodecamethylpentasiloxane, etc. and branched volatile methyl siloxane solvents such as 1,1,1,3,5,5,5-Heptamethyl-3-[(trimethylsilyl)oxyl]-trisiloxane, 1,1, 1,5,5,5-Hexamethyl-3,3, -bis[(trimethylsilyl)oxy]-trisiloxane, Pentamethyl[(trimethylsilyl)oxy]-cyclotrisiloxane.

The lubricant can be diluted in the medium in which the medium comprises from about 1 wt % to about 99.9 wt % of a mixture of the medium with the lubricant. The range of dilution can depend on the medium. For example, a water medium can be used from about 1 wt % to about 99.9 wt % and an alcohol medium such as isopropanol can be used from about 1 wt % to about 99.9 wt %. The lubricant can be applied to the bonded layer, undiluted or diluted, and by dip-coating, wiping, spraying (including aerosol spray), etc.

An exemplary formulation of the present disclosure can include one or more polymerizable silane monomers and/or siloxane monomers as the reactive component, an acid catalyst, e.g., HCl, phosphoric acid, acetic acid, and a solvent. Drying such a formulation polymerizes the monomers from exposed hydroxyl groups on the surface of the substrate to form an array of linear polysilanes or polysiloxanes or a combination thereof. By this technique, the array of linear polymers has ends covalently bound to the surface and opposite ends extending away from the surface and resemble a brush.

Advantageously, the formulations of the present disclosure can be applied to surfaces of ceramic or metal toilets, sinks, plumbing fixtures, surfaces of glass substrates including mirrors, windshields, windows in a building, a glass optical lens for a camera, surfaces composed of one or more polymers such as plastic sinks, toilets, surfaces of personal protective equipment such as gowns, face shields goggles, shoe covering and shoes and medical devices such as ostomy appliances, catheter, syringe, scalpel, endoscope lens, metal and plastics implants (e.g., orthopedic implants, dental implants, glaucoma implants), prostheses, etc.; automobile parts such as windshields, camera lens, lamp and sensing casings, mud flaps, car bodies; airplane parts such as windshield, airplane wings and bodies; marine parts such as submerged devices, cables, ships and boats; outdoor and indoor signage, bus step enclosures; reusable and disposable consumer containers and packaging such as for cosmetics, foods, hair and skin care products, such as shampoos, oral products including toothpaste, etc.

Many medical devices can benefit from the formulations and repellent coatings of the present disclosure including medical devices composed of polymeric surfaces. For example, an ostomy appliance (bag or pouch as they are commonly referred) can include a collection pouch and one or more ports including one or more outlet ports. Such ostomy appliances have surfaces typically made of one or more polymers that can be coated with formulations of the present disclosure to form one or more repellent coated surfaces. In one aspect of the present disclosure, a surface of an ostomy appliance, e.g., an inner surface, can include a repellent coating prepared by drying a formulation of the present disclosure on a material to form such a surface to substantially remove the solvent and to form a bonded layer on the surface. Further if a lubricant is included in the formulation or subsequently applied, the repellent coating further includes a lubricant layer stably adhered to the bonded layer formed from the lubricant. In addition, the surface of the substrate surface used to form the ostomy appliance can be treated to form reactive groups such as hydroxyl groups, such as by applying and removing an alcohol, or by oxygen plasma treatment, prior to applying and drying a formulations of the present disclosure.

### EXAMPLES

### Example 1: Volatile Organic Compound Exempt Solvent Formulations and Stability

In the following experiments, formulations that included various volatile organic compound (VOC) exempt solvents were compared for stability. For these experiments, smooth glass slides were used as substrates (such glass slides can be obtained from McMaster-Carr as 25 mm×75 mm microscope slides). The glass slides were cleaned by isopropanol. Formulations having the components and concentrations provided in Tables 1 and 2 below were applied to different glass slides by dip coating. The applied formulations were allowed to dry within 1-20 minutes under room temperature and atmospheric pressure to form coatings on the slides. The shelf-life and liquid repellency characterizations of coated glass samples formed by various formulations with different VOC exempt solvents are provided in Table 2 below.

TABLE 1
Formulation containing a VOC exempt solvent.

| Component | Approximate Concentration |
|---|---|
| Reactive Monomer: | 9.0 wt % |
| Dimethoxydimethylsilane | |
| Solvent: | 89.0 wt % |
| Various (see Table 2) | |
| Acid Catalyst: | 1.0 wt % |
| Sulfuric acid | |
| Lubricant: | 1.0 wt % |
| Silicone oil, 50 cSt | |

TABLE 2
Solvents and sliding angle on coated glass prepared from the formulations
listed in Table 1.

| Solvent | Viscosity | Evaporation of | Sliding | Shelf life |
|---|---|---|---|---|
| | (at 25° C.) | solvent | Angle (°) | (days) |
| Dodecamethyl | 2 cSt | Slow (~10 min) | 4 ± 1 | >180 |
| pentasiloxane | | | | |
| Octamethyl | 2.5 cSt | Medium (~5 min) | 3 ± 1 | <5 |
| cyclotetra siloxane | | | | (failed) |
| Hexamethyl | 0.65 cSt | Fast (~1 min) | 4 ± 1 | >180 |
| disiloxane | | | | |
| Acetone | 0.39 cSt | Fast (~1 min) | 5 ± 1 | >60 |
| | | | | color darkened |
| | | | | overtime |
| Propylene carbonate | 2.08 cSt | Slow (>10 min) | 90 (failed) | — |
| Dimethyl carbonate | 0.55 cSt | Fast (~2 min) | 90 (failed) | — |

Table 2 includes sliding angle (SA) data for coating surfaces prepared with formulations stored after the period of time listed in the table. Sliding angles were measured by placing a 20 μL water droplet on the coated surface of the substrate. The water used for the measurements was deionized. The substrates were subsequently tilted gradually from a horizontal position until the water droplet began to slide off the substrate. The angle (formed between horizontal and the flat tilted substrate) at which the water droplet began to slide was taken as the sliding angle. At least three sliding angle measurements were made and the averaged sliding angle provided in Table 2.

Average sliding angles were measured immediately after mixing the formulations and at subsequent storage periods. Formulations indicated with a shelf life greater than 180 days produced coatings having average sliding angles that did not change much over the storage period and were significantly less than 20 degrees. Formulations indicated with shelf life of less than 5 days produced coatings having sliding angles of around 90 degrees after the 5 days storage period. A sliding angle of about 70° or higher for a 20 μL water droplet on a coated surface is considered a non-repellent surface for these experiments.

As shown in Table 2, carbonate solvents such as propylene carbonate and dimethyl carbonate failed to provide a repellent coating on a substrate surface when formulations included these compounds as the solvent. Further, acetone as a solvent changed color from clear to a black color after 60 days of storage. In addition, a cyclic siloxane solvent (octamethylcyclotetrasiloxane) included in the formulation as the solvent resulted in a coating that had a sliding angle of about 90° after the formulation was stored for 5 days.

Based on observations with the linear volatile methyl siloxane solvents, it is believed that the only VOC exempt solvents tested that had sufficient stability in the tested formulations were the linear volatile methyl siloxane solvents. These solvents included dodecamethylpentasiloxane and hexamethyl disiloxane.

### Example 2: Repellant Coatings Prepared from Linear Volatile Methyl Siloxane Solvent and Isopropanol Solvent

Repellent coatings were formed directly on several polymeric substrates and other substrate surfaces. For these experiments, formulations having the components and concentrations provided in Table 3 and Table 4 below were applied to different substrates. The substrates and respective pretreatments were listed in Table 5 below. Sliding angles were measured by placing a 15 μL water droplet (deionized water) on the coated surface of the substrate and measurements were made as described in the previous experiments of Example 1. (Although a 15 μL water droplet was used for these experiments rather than a 20 μL water droplet, the sliding angle values using the smaller 15 μL water droplet would be about the same or greater than if the measurements were made with a 20 μL water droplet. Stated differently, the sliding angle for a 20 μL droplet would be expected to be lower than that of a 15 μL droplet on the same surface.)

Samples pre-treated by oxygen plasma were carried out by an oxygen plasma treatment which took at least 15 seconds using a Harrick Plasma cleaner PDC-001 at high RF power (30 W) and 300 mTorr vacuum. As noted in Table 5 below, some samples were pre-treated by cleaning with isopropanol instead of oxygen plasma. Titanium was heated to 250° C. for 20 minutes on a hotplate to prepare the surface before coating. Formulations having the components and concentrations provided in Tables 3 and Table 4 below were applied to different samples by wiping with paper towels containing the formulations. Once applied, the formulations on the surfaces were allowed to evaporate by drying for at least 1 minute at room temperature and atmospheric pressure to form the coatings.

TABLE 3
Formulation containing a VOC solvent and a lubricant.

| Component | Approximate Concentration |
|---|---|
| Reactive Monomer: | 9.0 wt % |
| Dimethoxydimethylsilane | |
| Solvent: | 89.0 wt % |
| Isopropyl alcohol | |
| Acid Catalyst: | 1.0 wt % |
| Sulfuric acid | |
| Lubricant: | 1.0 wt % |
| Silicone oil, 50 cSt | |

TABLE 4
Formulation containing a VOC exempt solvent and a lubricant.

| Component | Approximate Concentration |
|---|---|
| Reactive Monomer: | 9.0 wt % |
| Dimethoxydimethylsilane | |
| Solvent: | 89.0 wt % |
| Hexamethyldisiloxane | |
| Acid Catalyst: | 1.0 wt % |
| Sulfuric acid | |
| Lubricant: | 1.0 wt % |
| Silicone oil, 50 cSt | |

TABLE 5
Comparison of liquid repellency measurements of different coated
substrates using formulations including isopropanol and
hexamethyldisiloxane as solvents.

| | | Table 3 | Table 4 (with |
|---|---|---|---|
| | | (with VOC | VOC exempt |
| | | solvent) | solvent) |
| | Pre-treatment | Sliding angle | Sliding angle |
| Substrate | of surface | (degree) | (degree) |
| Polyurethane | Oxygen plasma | 11 ± 2 | 12 ± 2 |
| EVA | Oxygen plasma | 17 ± 3 | 16 ± 2 |
| (poly (ethylene-vinyl | | | |
| acetate) film from | | | |
| USI, Inc.) | | | |
| Polypropylene | Oxygen plasma | 19 ± 2 | 20 ± 3 |
| High density | Oxygen plasma | 15 ± 3 | 16 ± 3 |
| polyethylene (HDPE) | | | |
| Ceramic | Oxygen plasma | 10 ± 2 | 8 ± 2 |
| Ceramic | Isopropanol | 10 ± 3 | 12 ± 2 |
| | alcohol clean | | |
| Titanium | Heat treatment | 11 ± 3 | 12 ± 3 |
| | 250° C., 20 min | | |

As shown in Table 5 above, each coating formed from a linear volatile methyl siloxane solvent (hexamethyldisiloxane) (Table 3) on the various surfaces of the substrates had an average sliding angle of less than about 20 degrees. Further, the repellent coatings formed from formulations including a linear volatile methyl siloxane solvent (hexamethyldisiloxane) were as good as those formed from a formulation including isopropanol as a solvent. The data show that using a formulation based on a linear volatile methyl siloxane solvent can form repellant coatings on a wide variety of surfaces and with good repellent characteristics.

### Example 3: Formulation with Fluorinated Alkyl Silane as Reactive Component and a Linear Volatile Methyl Siloxane Solvent

In the following experiments, a formulation including a fluorinated alkyl silane and linear volatile methyl silane solvent was tested. The formulation is listed in Table 6 below and the results are provided in Table 7 below.

For these experiments, samples were pre-treated by oxygen plasma or isopropanol alcohol. The pre-treatment details were similar to those listed in Example 2 pre-treated by oxygen plasma or isopropanol alcohol. The formulations were applied to different samples by wiping with paper towels containing the formulations. Once applied, the formulations on the surfaces were left to dry for at least 1 minute at room temperature and atmospheric pressure to form the coatings.

Then a lubricant, Krytox 103 (i.e., a perfluoropolyether), was applied to the bonded layer by wiping with paper towels containing the lubricant (undiluted) at room temperature and atmospheric pressure to form a lubricant layer stably adhered to the fluorinated silane bonded layer as the repellent coating. Sliding angles were measured by placing 15 μL water or oil droplets on the coated surface of the substrate and measurement as described in the previous experiments.

TABLE 6
Formulation including a fluorinated alkyl silane and linear volatile
methyl silane solvent.

| Component | Approximate Concentration |
|---|---|
| Reactive Monomer: | 9.0 wt % |
| Heptadecafluoro-1,1,2,2-tetrahydrodecyl) | |
| trimethoxysilane | |
| Solvent: | 89.0 wt % |
| Hexamethyldisiloxane | |
| Acid Catalyst: | 1.0 wt % |
| Sulfuric acid | |

TABLE 7
Liquid repellency measurements of different substrates having a
fluorinate repellent coating.

| | | Sliding angle | Sliding angle |
|---|---|---|---|
| | | of Water | of Olive |
| | | Droplet | Oil Droplet |
| Different surfaces | Pre-treatment | (degree) | (degree) |
| Ceramic | Isopropanol | 13 ± 2 | 15 ± 4 |
| | alcohol clean | | |
| Glass | Isopropanol | 11 ± 3 | 15 ± 3 |
| | alcohol clean | | |
| High density | Oxygen plasma | 12 ± 2 | 12 ± 2 |
| polyethylene (HDPE) | | | |
| Polyurethane | Oxygen plasma | 9 ± 2 | 10 ± 3 |
| EVA | Oxygen plasma | 12 ± 3 | 12 ± 3 |
| (poly (ethylene-vinyl | | | |
| acetate) film from | | | |
| USI, Inc.) | | | |

As shown in Table 7 above, the repellent coating formed from a fluorinated alkyl silane reactive component in a formulation including a linear volatile methyl silane as the solvent and with a perfluorinated lubricant layer thereon showed a sliding angle of less than 20 degrees to both aqueous (water) and non-aqueous (olive oil) liquids. Thus showing that repellent coatings can be formed from fluorinated components using a linear volatile methyl siloxane solvent and that such coatings repel both aqueous and oil substances (i.e., omniphobic).

### Example 4: Additional Formulations

Smooth glass slides were cleaned with isopropanol followed by the application of a coating formulation shown in Table 8 below by wiping the glass slides with a paper towel containing the particular formulation. All formulations listed in Table 8 below included 5 vol % of the lubricant, Silicone oil, 50 cSt. The concentrations of acid catalyst and reactive component were also listed. The solvent made up the rest of the formulation.

Once a formulation was applied to a glass substrate surface, the formulation on the surface was left to dry at room temperature and atmospheric pressure to form the coatings. The shelf-life and liquid repellency characterizations of coated glass samples formed by various formulations with different VOC exempt solvents and different acid catalysts at different amounts are provided in Table 8 below.

TABLE 8
Formulations with varying components and amounts and liquid repellency
characteristics.

| | | | Contact | Sliding | ^{†††}Shelf | |
|---|---|---|---|---|---|---|
| Solvent / | | ^{†}Reactive | Angle | Angle | life | ^{††}VOC |
| Carrier | Acid Catalyst | Component | (°) | (°) | (days) | (%) |
| Dodecamethyl | Acetic acid (2 | 1 vol % | 100 | 17 ± 2 | >60 | <1 |
| pentasiloxane | wt %) | | | | | |
| Dodecamethyl | Phosphoric | 1 vol % | 101 | 15 ± 3 | >60 | <1 |
| pentasiloxane | acid (2 wt %) | | | | | |
| Polydimethyl | Phosphoric | 10 vol % | 96 | 18 ± 2 | >107 | 1.14 |
| siloxane (3 cSt) | acid (10 wt %) | | | | | |
| Hexamethyl | Phosphoric | 10 vol % | 94 | 19 ± 2 | >142 | 5.34 |
| disiloxane | acid (10 wt %) | | | | | |
| Dodecamethyl | ^{††††}Citric acid | 1 vol % | 94 | 15 ± 2 | <14 | <1 |
| pentasiloxane | in 1-propanol | | | | | |
| | solution (1 | | | | | |
| | vol %) | | | | | |
| Dodecamethyl | Citric Acid in | 1 vol % | 99 | 17 ± 2 | <2 | |
| pentasiloxane | 1-propanol | | | | | |
| | solution (2 | | | | | |
| | vol %) | | | | | |
| Polydimethyl | Citric Acid in | 1 vol % | 96 | 15 ± 2 | <2 | |
| siloxane (3 cSt) | 1-propanol | | | | | |
| | solution (2 | | | | | |
| | vol %) | | | | | |

^{†}The reactive component was dimethyl dimethoxy silane.
^{††}Volatile Organic Compound (VOC) determinations of the various formulations were made using the California Air Resources Board (CARB) 310 protocol (August 2014).
^{†††}Average sliding angles were measured immediately after mixing the formulations and at subsequent storage periods. Formulations indicated with a shelf life greater than 60 days produced coatings having average sliding angles that did not change much over the storage period and were significantly less than 35 degrees and typically less than about 20 degrees. Formulations indicated with shelf life of less than 14 days and less than 2 days produced coatings having sliding angles of around 90 degrees after the 14 days or 2 days storage period and were considered non-repellent.
^{††††}50 g of 1-Propanol was used to dissolve 100 g of citric acid to form a citric acid solution. The citric acid solution accounted for 1 vol % and 2 vol %, respectively, of the final formulation.

Sliding angles were measured by placing 15 μL DI water or droplets on the coated surface of the substrate and measurement as described in the previous experiments.

Table 8 above shows that the concentration of acid catalyst can range from 1 wt % to 10 wt % with a methyl siloxane solvent in a formulation and such formulations give comparable coating performance. Table 8 also shows formulations of the present application can have a low VOC level, as determined using the California Air Resources Board (CARE) 310 protocol (August 2014), such as a VOC level of less than 6%, e.g., even less than 2%. Table 8 above also shows that when a significant amount of a lower primary alcohol (1-propanol) is included as part of the solvents, the formulations had a shelf-life of less than 14 days.

Only the preferred embodiment of the present invention and examples of its versatility are shown and described in the present disclosure. It is to be understood that the present invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances, procedures and arrangements described herein. Such equivalents are considered to be within the scope of this invention, and are covered by the following claims.

What is claimed is:

1. A process of forming a repellant coating on a medical device from a formulation comprising: (i) one or more reactive components that can form a bonded layer on a surface in which the bonded layer comprises an array of compounds each compound having one end bound to the surface and an opposite end extending away from the surface; (ii) an acid catalyst; (iii) a solvent comprising a non-cyclic volatile siloxane; and optionally (iv) a lubricant, the process comprising:
drying the formulation on a surface of the medical device to substantially remove the solvent and to form a bonded layer on the surface.

2. The process of claim 1, wherein the formulation is dried in air and at atmospheric pressure.

3. The process of claim 1, further comprising treating the surface of the device with an oxygen or air plasma to generate hydroxyl groups on the surface of the device followed by applying the formulation on the surface and drying the formulation to form the repellent coating on the surface.

4. The process of claim 1, wherein the formulation includes a lubricant and the lubricant includes a silicone oil or a mineral oil or a plant oil or any combination thereof having a viscosity of at least about 5 cSt as measured at 25° C.; and drying the formulation on the surface of the device to substantially remove the solvent forms the bonded layer on the surface with the lubricant formed as a lubricant layer stably adhered to the bonded layer.

5. The process of claim 4, further comprising applying the lubricant or a different lubricant to the bonded layer.

6. The process of claim 1, wherein the formulation does not include a lubricant and the process further comprises applying a lubricant to the bonded layer to form a lubricant layer stably adhered to the bonded layer.

7. The process of claim 6, wherein the lubricant is a fluorinated oil.

8. A formulation comprising: (i) one or more reactive components that can form a bonded layer on a surface in which the bonded layer comprises an array of compounds each compound having one end bound to the surface and an opposite end extending away from the surface; (ii) an acid catalyst; (iii) a solvent comprising a non-cyclic volatile siloxane; and optionally (iv) a lubricant.

9. The formulation of claim 8, wherein the one or more reactive components are one or more dialkyl di-alkoxy silanes.

10. The formulation of claim 8, wherein the formulation includes the lubricant and wherein the lubricant is a silicone oil or a mineral oil or a plant oil or any combination thereof having a viscosity of at least about 5 cSt as measured at 25° C.

11. The formulation of claim 8, wherein the one or more reactive components are one or more fluoroalkyl silanes.

12. The formulation of claim 8, wherein the solvent comprises at least 90 wt % of the non-cyclic volatile siloxane based on a total weight of the solvent.

13. The formulation of claim 8, wherein the solvent is a linear or a branched volatile methyl siloxane solvent or combinations thereof.

14. The formulation of claim 8, wherein the formulation has a stable shelf-life of at least one month of a storage period.

15. A process of forming a repellent coating on a surface of a substrate from a formulation according to claim 8, the process comprising:
drying the formulation on a surface of a substrate to substantially remove the solvent and to form a bonded layer on the surface and, if present, the lubricant forms a lubricant layer stably adhered to the bonded layer.

16. The process of claim 8, wherein the surface of the substrate comprises glass, porcelain, metal and/or a polymer.

17. The process of claim 8, wherein the substrate is a toilet, sink, mirror, window, or stove.

18. An ostomy appliance having a repellent coating on a surface thereof obtained from claim 8.
